(19) United States
(12) Patent Application Publication
(10) Pub. No.: US 2025/0255754 A1
(43) Pub. Date: Aug. 14, 2025

(54) HYDRO-DEGRADABLE FLUID ABSORBENT

(71) Applicant: ANABIO TECHNOLOGIES PRIVATE LIMITED, Karnataka (IN)

(72) Inventors: Mithun A. SHAH, Bengaluru, Karnataka (IN); Abhilash KULKARNI, Bengaluru, Karnataka (IN); Anshul AGRAWAL, Bengaluru, Karnataka (IN); Ruchi ARADHANA, Bengaluru, Karnataka (IN)

(21) Appl. No.: 18/856,347

(22) PCT Filed: Jun. 13, 2023
(86) PCT No.: PCT/IB2023/056067

§ 371 Date: Oct. 11, 2024

(30) Foreign Application Priority Data
Apr. 13, 2022 (IN) 202241022228

(51) Int. Cl. A61F 13/15; A61F 13/511; A61F 13/539

(52) U.S. Cl. CPC A61F 13/15252; A61F 13/15211; A61F 13/15577; A61F 13/51121; A61F 13/539; A61F 2013/15235; A61F 2013/15869; A61F 2013/15878

(57) ABSTRACT

A hydro-degradable fluid absorbent product and its componentry, construction, and a method of manufacturing the same. The hydro-degradable fluid absorbent includes a top sheet formed of hydro-degradable layer, wherein the top sheet along with the edges is coated with hydrophobic coating that covers 0-80% of the said sheet and 100% of its edges, an absorbent core, optionally with super absorbent polymer, a bottom sheet including at least one water soluble film characterised by a tie layer which is coated on water-soluble film followed by moisture barrier coat. The method of manufacturing includes assembling a top sheet formed by a hydro-degradable layer, an absorbent core, optionally with super absorbent polymer, the bottom sheet including at least one water soluble film characterized by a tie layer, and bonding the top sheet, the bottom sheet with the absorbent core sandwiched in between by utilizing appropriate industrial bonding techniques.

### TECHNICAL FIELD

The present invention generally relates to hydro-degradable fluid absorbent product for use as pantiliners, sanitary napkins, incontinence pads, packaging, wound dressing and the like. In particular, the present invention relates to hydro-degradable fluid absorbent product's componentry, construction and a method of manufacturing the same.

### BACKGROUND

Disposability of the feminine hygiene products is one of the biggest issues as it poses a biohazard risk. Sorting and segregation of these products in waste management require human contact which pose social stigma and the biohazard problem. Flushing of soiled sanitary napkins in toilets can potentially clog the drain pipes and lead to blockage of sewage pipelines. Biodegrading these plastic absorbent products than allowing to degrade naturally has been explored exponentially as the plastics used in absorbent materials takes hundreds of years to decompose naturally. Research in the field of sanitary waste disposal led to the advent of water dispersible materials that can be flushed into the toilets directly after use thereby incorporating water dispersible materials in absorbent products such as diapers, sanitary pads and pantyliners.

U.S. Pat. No. 5,830,201A describes an envelope which has inner and outer walls which are secured together at their outer periphery to form an inner cavity. The outer wall is comprised of a hydrophobic outer layer together with a hydrophilic inner layer. The inner wall is comprised of a hydrophobic outer layer together with a hydrophilic inner layer.

US 2006/0154054A1 describes a flushable bodily liquid absorbent composite product, having a bodily liquid absorbent core and a backing layer applied to a garment side of the core. The backing layer is readily soluble in cold water and has a water impervious layer on its core side and a water resistant layer on its garment facing side.

Some of the problems associated with the prior art product are,
a) the water soluble film of bottom layer which disintegrates during peel off. When the water soluble bottom layer is exposed towards the garment side, the bottom layer weakens due to absorption of water vapour in the presence of humidity leading to weakening of structure and later disintegration during peel off thereby sticking to the undergarment.
b) the adhesive used in the bottom layer doesn't come off easily from the undergarment while removal and tends to be attached to the underwear. Due to the presence of humidity, not only the bottom layer gets weaken, the position adhesive also sticks to the underwear.
c) Environmental humidity from outside affecting the absorbent article during peel off of the hydro-degradable absorbent article.
d) Wettability issue of the hydro-degradable top sheet during usage of the sanitary pads due to high absorption capacity of the top sheet which needs to be addressed.

Accordingly there is a need for an efficient water dispersible/hydro-degradable fluid absorbent which obviates the foregoing and other limitations and disadvantages of prior art products. Despite the availability of flushable diapers and sanitary pads in the prior art, there is no availability of a suitable solution to the problems of prior art.

### SUMMARY OF THE INVENTION

The present invention provides a hydro-degradable fluid absorbent product's componentry, construction and a method of manufacturing the same.

According to an embodiment of the present disclosure there is provided a hydro-degradable fluid absorbent comprising:
a) a top sheet formed of hydro-degradable layer, wherein the top sheet along with the edges is coated with hydrophobic coating that covers 0-80% of the said sheet and 100% of its edges;
b) an absorbent core, optionally with super absorbent polymer;
c) a bottom sheet comprising at least one water soluble film characterized by a tie layer which is coated on water-soluble film followed by moisture barrier coat.

According to another embodiment of the present disclosure there is provided a hydro-degradable fluid absorbent wherein the bottom sheet is constructed in a way such that the water-soluble film is towards the adsorbent core and moisture barrier layer is towards the garment side and vice-versa with the tie layer sandwiched in between.

According to another embodiment of the present disclosure there is provided a hydro-degradable fluid absorbent wherein the bottom sheet comprising at least one water soluble film characterized by a tie layer coated on both sides of water-soluble film followed by moisture barrier coat on both sides with variation of thickness on each side.

According to another embodiment of the present disclosure there is provided a hydro-degradable fluid absorbent wherein the bottom sheet comprising at least one water soluble film is characterized by a tie layer which is coated as alternate pattern of strips with coated and uncoated zones on water-soluble film followed by moisture barrier coat on top of coated alternate strips of the tie layer such that the said coating is carried out on both sides of the film. The coating on other side of the film is done on the area which is uncoated on the opposite side.

According to yet another embodiment of the present disclosure there is provided a hydro-degradable fluid absorbent wherein the bottom sheet comprising at least one water soluble film characterized by a tie layer which is coated as alternate pattern of strips of coat on water-soluble film followed by moisture barrier coat on top of coated alternate strips of tie layer and the other side of the film is coated with a tie layer in entirety, wherein, the tie-layer coated in entirety side of water-soluble film is towards the core and the coated moisture barrier layer towards the garment side and vice-versa.

According to yet another embodiment of the present disclosure there is provided a hydro-degradable fluid absorbent wherein the bottom sheet comprising at least one water soluble film characterized by a tie layer which is coated as alternate pattern of strips of coat on water-soluble film followed by moisture barrier coat on top of coated alternate strips of tie layer; the other side of the water soluble film is coated with a tie layer in entirety, followed by moisture barrier coat, wherein the tie layer coated alternate pattern of strips of coat side of the water-soluble film is towards the core and the coated moisture barrier layer towards the garment side and vice-versa.

In an aspect of the present disclosure, there is provided a hydro-degradable fluid absorbent wherein said at least one water soluble film of the bottom sheet comprises two sheets of water soluble film.

According to further embodiment of the present disclosure, there is provided a hydro-degradable fluid absorbent wherein the bottom sheet comprising of two sheets of water-soluble film characterized in that each film further comprises of a tie layer which is coated on water-soluble film followed by moisture barrier coat, such that the two sheets of film have water soluble layers facing towards each other, resulting in moisture barrier coat of one layer is towards the adsorbent core and of other layer is towards the garment side.

In an aspect of the present invention, the bottom sheet comprising of two sheets of water-soluble film characterized in that either both inner and outer sheet are of same width or the width of the inner sheet is less than the width of the outer sheet, such that both sheets or only one sheet (outer sheet) of film of the bottom sheet are heat sealed or glued with top sheet respectively, for construction of hydro-degradable fluid adsorbent. Such construction allow the center being more robust and provide a better barrier and sealing while in use and additionally resulting in faster opening of the sealed edges when exposed to water.

According to another embodiment of the present invention there is provided a method of manufacturing a hydro-degradable fluid absorbent comprising:
assembling a top sheet formed by a hydro-degradable layer, an absorbent core optionally with super absorbent polymer, a bottom sheet comprising at least one water soluble film characterized by a tie layer which is coated on water-soluble film followed by moisture barrier coat prepared according to any of the embodiments as aforesaid and bonding the top sheet and the bottom sheet with the absorbent core sandwiched in between by utilizing appropriate industrial bonding techniques such as thermal bonding, hot melt adhesives, water soluble adhesives, ultrasonic bonding, etc.

### BRIEF DESCRIPTION OF THE DRAWINGS

FIG. 1: Exploded perspective view of the hydro-degradable fluid absorbent.

FIG. 2: Exploded planar view of the hydro-degradable fluid absorbent.

FIG. 3: Exploded Planar View with hydrophobic coating on top sheet of the hydro-degradable fluid absorbent.

FIG. 4A and 4B: Top view of top sheet along with the edges coated with hydrophobic coating that covers 0-80% of the said sheet and 100% of its edges;

FIG. 5: Bottom View of the hydro-degradable fluid absorbent with the absorbent core visible from the peel.

FIG. 6: Macroscopic View of different layers of bottom sheet of the hydro-degradable fluid absorbent.

FIG. 7: Bottom perspective view showing visible layers of bottom sheet of the hydro-degradable fluid absorbent.

FIG. 8A, 8B, 8C and 8D: Sectional view of different layers of bottom sheet of the hydro-degradable fluid absorbent.

FIG. 9 Flowchart illustrating method of manufacturing of the hydro-degradable fluid absorbent.

### DETAILED DESCRIPTION OF THE INVENTION

The following description with reference to the accompanying drawings is provided to assist in a comprehensive understanding of exemplary embodiments of the invention. It includes various specific details to assist in that understanding but these are to be regarded as merely exemplary.

The terms and words used in the following description and claims are not limited to the bibliographic meanings, but are merely used by the inventor to enable a clear and consistent understanding of the invention. Accordingly, it should be apparent to those skilled in the art that the following description of exemplary embodiments of the present invention are provided for illustration purpose only and not for the purpose of limiting the scope of the invention as defined by the appended claims and their equivalents.

The present invention is described in connection with such embodiments, but the invention is not limited to any embodiment. The scope of the invention is limited only by the claims and the invention encompasses numerous alternatives, modifications and equivalents. Numerous specific details are set forth in the following description in order to provide a thorough understanding of the invention. These details are provided for the purpose of example and the invention may be practised according to the claims without some or all of these specific details. For the purpose of clarity, technical material that is known in the technical fields related to the invention has not been described in details so that the invention is not unnecessarily obscured.

The present invention relates to hydro-degradable absorbent product ideally suited for sanitary uses, particularly for the absorption of aqueous body fluids. The product comprises a highly porous, super absorbent, degradable fibrous core and a bottom sheet of specific construction attached to an absorbent core. In the preferred embodiment, the absorbent product also comprises a top sheet that is water-permeable and degradable. Certain parts of absorbent product are disintegrable, while certain other parts are degradable which allow the absorbent to open up when disposed in water.

In an embodiment of the present invention, there is provided a hydro-degradable fluid absorbent comprising:
a) a top sheet formed of hydro-degradable layer, wherein the top sheet along with the edges is coated with hydrophobic coating that covers 0-80% of the said sheet and 100% of its edges;
b) an absorbent core, optionally with super absorbent polymer;
c) a bottom sheet comprising of at least one sheet of-water-soluble film characterized by a tie layer which is coated on water-soluble film followed by moisture barrier coat.

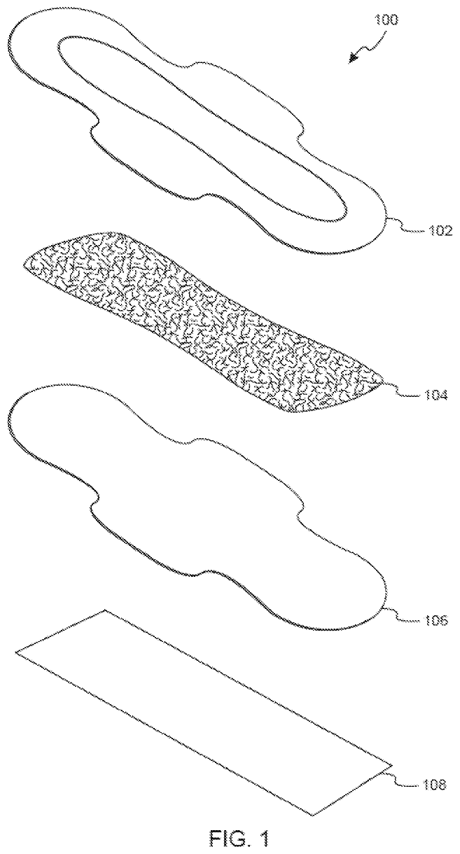

According to an embodiment of the present invention, the bottom sheet comprising of a single sheet of water-soluble film characterized by a tie layer which is coated on water-soluble film followed by moisture barrier coat, wherein the water-soluble film is towards the adsorbent core and moisture barrier coat is towards the garment side and vice-versa with the tie layer sandwiched in between. FIG. 1 illustrates an exploded perspective view of the hydro-degradable fluid absorbent (100), comprising a top sheet (102), an absorbent core (104), a bottom sheet (106) and a release liner (108) according to an embodiment of the present invention as disclosed herein.

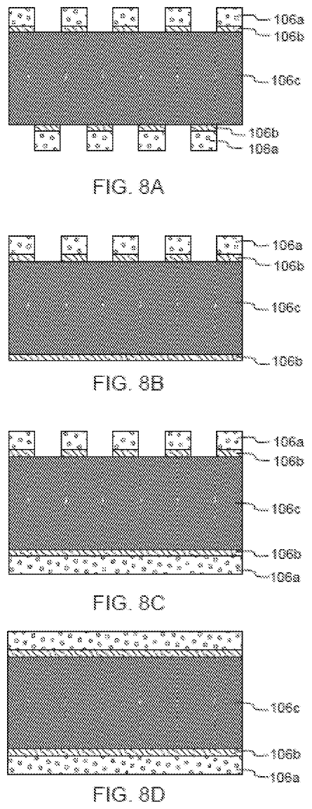

According to another embodiment of the present disclosure, the bottom sheet comprising of a tie layer which is coated as alternate pattern of strips with coated and uncoated zones on water-soluble film followed by moisture barrier coat on top of coated alternate strips of the tie layer such that the said coating is carried out on both sides of the film (as depicted in FIG. 8A). The coating on other side of the film is done on the area which is uncoated on the opposite side.

According to yet another embodiment of the present disclosure, the bottom sheet comprising of two sheets of water-soluble film characterized in that each film further comprises of a tie layer which is coated on water-soluble film followed by moisture barrier coat, wherein the bottom sheet is constructed such that the two sheets of film have water soluble layers facing towards each other, resulting in moisture barrier coat of one layer is towards the adsorbent core and of other layer is towards the garment side.

The term “degradable” as used herein means that the product is able to breakdown via hydrolysis and/or aqueous products in concentrations generally harmless to the environment. The term “disintegrate” as used herein means that the product is able to break up into small parts as the result of impact or decay and also to lose strength or cohesion and gradually fail.

The wood pulp in the top sheet and absorbent core, chemicals such as polyvinyl alcohol, dissolve/disintegrate out of the structures of the present invention, may biodegrade and/or; chemically degradable via simple environment/compost waste treatment conditions or by recycle or sewage chemical treatment.

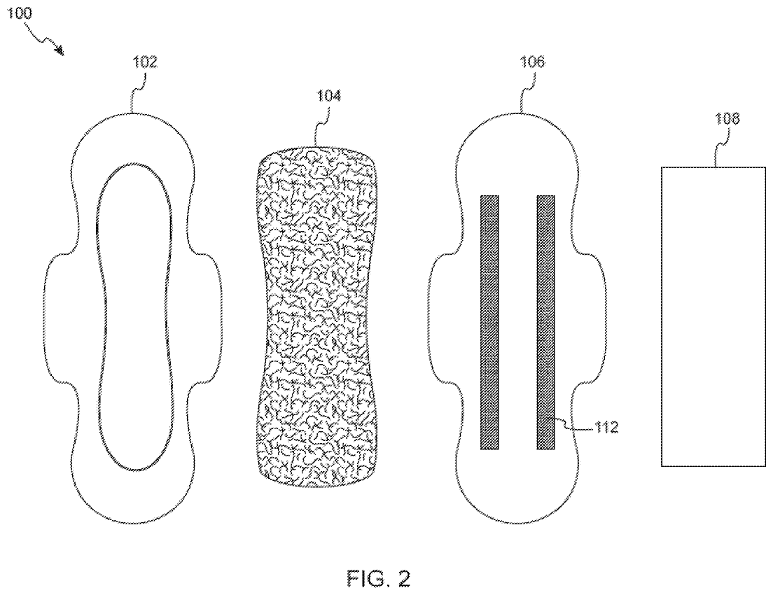

The terms “top sheet”, “bottom sheet” and “core” as used herein, have the meaning conventional in the sanitary napkin art. Specifically, the top sheet is the layer adjacent to the human skin; the back sheet is the layer remote from the skin; and the core is the batting material sandwiched between top sheet and bottom sheet. The absorbent product may contain more than three layers, but these three are the significant component of the fluid absorbent product of the present invention. FIG. 2 illustrates an exploded planar view of the hydro-degradable fluid absorbent (100) showing different layers according to an embodiment of the present invention as disclosed herein.

The top sheet used in sanitary pad has two important functions: Collect the discharge & pass it to the absorbent core, and feel comfortable to wearer in terms of wettability, durability, and softness. In currently available sanitary pads, majority of the top sheet is made of synthetic fiber such as polypropylene because of low density and high tensile strength. But these synthetic fibers are non-biodegradable and the discarded pads remains in the environment for long time. Also, pads made of these synthetic fibers cannot be flushed in the toilet because of high strength, it will not break in the sewer system and will clog the drainage leading to expensive maintenance.

In an aspect of the present invention of hydro-degradable fluid absorbent wherein the top sheet comprises of a highly absorptive cellulosic material.

For a flushable sanitary pad, the top sheet should not only collect discharge & pass it to absorbent core, and feel comfortable to wearer, but also disintegrate post disposal in the toilet. The top sheet properties should be such that it survives during the use and when discarded/flushed, disintegrate or break in the toilet and/or plumbing/sewage system.

In another aspect of the present invention of hydro-degradable fluid absorbent wherein the top sheet is a spun lace fabric made of biodegradable fibers such as wood pulp, viscose, etc with a GSM of 30-80.

The top sheet made of 100% wood pulp is very hydrophilic in nature and can absorb discharge up to 100-500% of its weight. Because of high absorption, the top sheet will become very wet during use leading to discomfort to the wearer. The other issue when it becomes very wet is the durability of the sheet, as it can disintegrate during the use.

In another aspect of the present invention of hydro-degradable fluid absorbent wherein the top sheet along with the edges is coated with hydrophobic coating that covers 0-80% of top sheet and 100% of its edges. The hydrophobic coating comprises of hydrophobic materials.

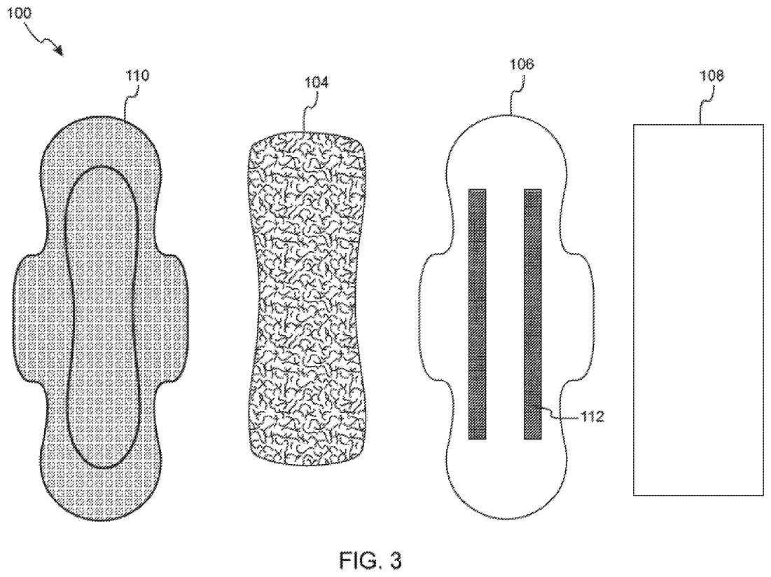

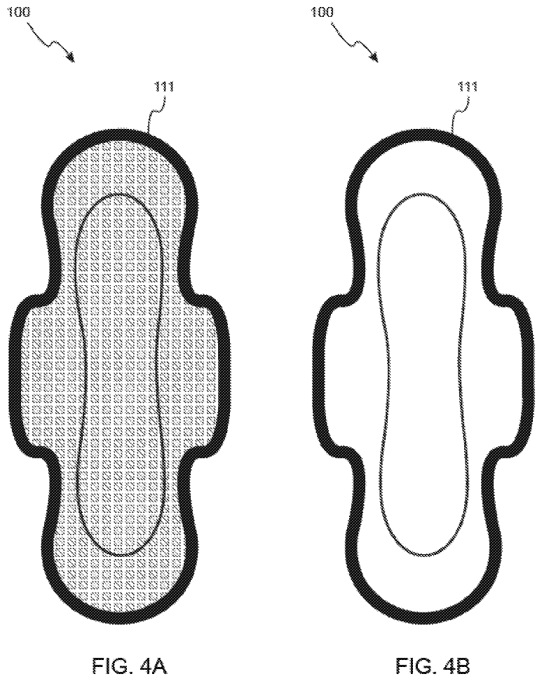

In yet another aspect of the present invention of hydro-degradable fluid absorbent wherein the hydrophobic coating comprises of lanolin. The other reason to use natural wax material is because of its non-skin irritant properties. The coating is applied to top sheet using printing/coating technologies known in the art. The surface area covered with the hydrophobic coating should be such that the top sheet absorbs the discharge and pass it to the absorbent core and at the same time feels dry and comfortable to the wearer. Additionally, this surface coating reduces the contact area of the top sheet with the skin which improves the abrasion resistance of the top sheet during use. As shown in FIG. 3, which depicts an exploded planar view with hydrophobic coating (110) represented as small squares on top sheet of the hydro-degradable fluid absorbent. The gap between the small squares of wax coating allows disintegration of the top sheet. FIG. 4A and 4B illustrates top view of top sheet along with the edges coated with hydrophobic coating that covers 0-80% of top sheet and 100% of its edges (111).

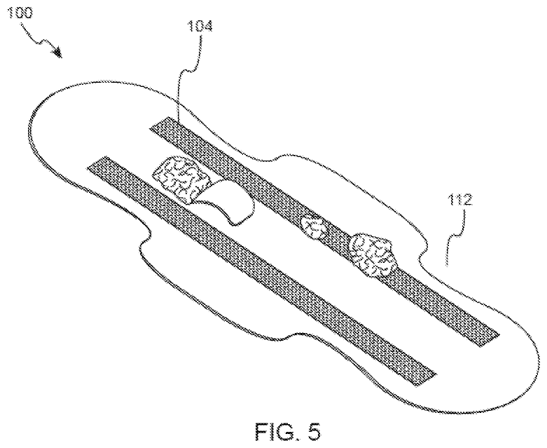

In another aspect of the present invention of hydro-degradable fluid absorbent wherein the absorbent core of the sanitary pad is generally made up of cellulosic pulp optionally with SAP Recently, super absorbent polymer (SAP) is air laid with pulp and used to increase absorbency. The position of this polymer is important as it may and restrict absorption of fluid, so the SAP polymer is affixed to a tissue and placed between the pulp and back sheet. The advantage that SAP offers is the absorption capacity i.e., it can absorb up to 100 times its own weight unlike cellulosic fibers. FIG. 5 illustrates a bottom view of the hydro-degradable fluid absorbent (100) with the absorbent core (104) visible from the peel according to an embodiment of the present invention as disclosed herein.

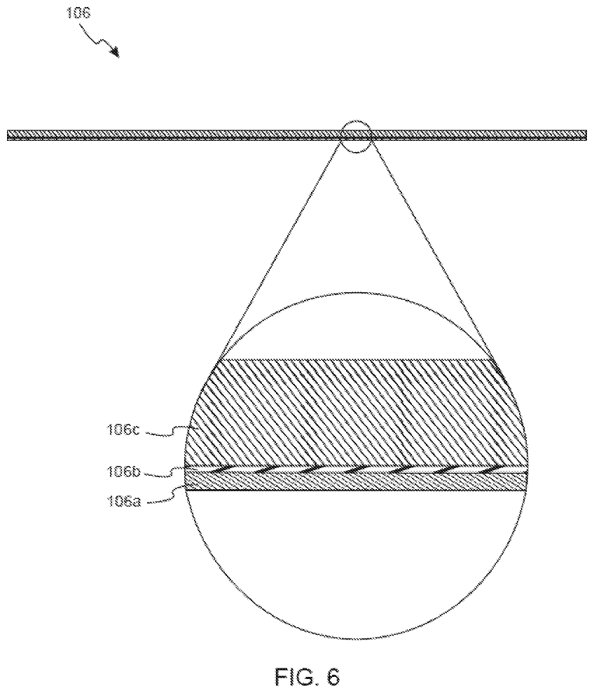

FIG. 6 depicts a macroscopic view of different layers of bottom sheet (106) of the hydro-degradable fluid absorbent (100). The bottom sheet is made up of water-soluble film example partially hydrolysed polyvinyl alcohol (PVOH).

In other aspect of the present invention of hydro-degradable fluid absorbent wherein the PVOH is cold water soluble and is used is in the range of 10-40 GSM.

The moisture barrier layer is coated on one side of the bottom layer using chemistries such as Polyvinylidene chloride (PVDC) to make it impervious to water and moisture on one side. The basis weight of the PVDC is around 1-15 GSM.

In another aspect of the present invention of hydro-degradable fluid absorbent wherein the tie layer is made of nitrocellulose whose thickness varies between 0.1 to 4 GSM. The tie layer constituted of nitrocellulose based chemistry (Examples include, Hubergroup™ products such as GR 2K OP TRPT INK HG HR-70GLR119C3, HARDNER 2K HG HR-70GHR959C3) is used to improve the adhesion between the PVOH and PVDC. Further, this tie layer acts as an anchoring layer for PVDC to PVOH which in turn controls the elasticity and brittleness of the coated material. The tie layer is first coated onto the PVOH layer and then, PVDC layer is coated on top of the tie layer.

In an embodiment of the present invention of hydro-degradable fluid absorbent wherein, that the bottom sheet can be coated with tie layer and PVDC in pattern of strips of various width. The pattern resembles alternate strips of coat where one strip is coated and adjacent is uncoated and continues in the same till the width of the film. The biggest advantage that these patterns offer is the disintegration when the product is flushed down. The flushed absorbent product easily disintegrates when exposed to water as the uncoated substrates dissolves faster.

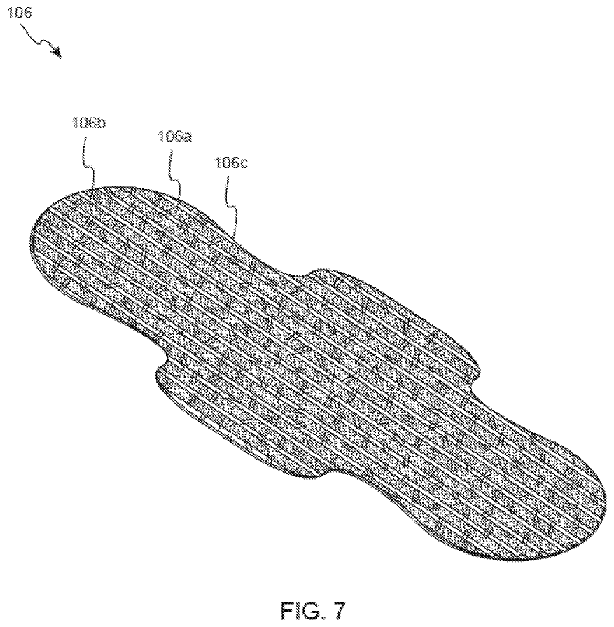

FIG. 7 depicts a bottom perspective view showing the PVDC (106a), the tie layer (106b) and the PVOH (106c) of bottom sheet (106) of the hydro-degradable fluid absorbent.

In an aspect of the present invention, the bottom sheet comprising of two sheets of water-soluble film characterized in that either both inner and outer sheet are of same width or the width of the inner sheet is less than the width of the outer sheet, such that both sheets or only one sheet (outer sheet) of film of the bottom sheet are sealed or bonded with top sheet respectively, for construction of hydro-degradable fluid adsorbent. These two sheets of bottom sheet can either be boned together only at the edges or throughout the surface by applying appropriate industrial bonding techniques. Such construction allow the center being more robust and provide a better barrier and sealing while in use and additionally resulting in faster opening of the sealed edges when exposed to water.

In another aspect of the present invention of hydro-degradable fluid absorbent, wherein the top sheet is bonded to the bottom sheet by suitable industrial bonding techniques.

In all the aforesaid embodiments of hydro-degradable fluid absorbent wherein, the bottom sheet comprising of at least one sheet water-soluble film characterized by a tie layer which is coated on water-soluble film followed by moisture barrier coat offers better structural integrity during the use and removal of the pad upon usage. To obtain desirable durability of the hydro-degradable fluid absorbent, the coating weight of the tie layer and moisture barrier layer used is preferably in the range of 4-15 GSM. Further advantages are a) the coated side does not disintegrate during peel off because the barrier layer is on the outside which offers protection against not only humidity but also liquids; b) the glue on the coated side does not peel and stick to underwear; c) the bottom layer delaminates as the water soluble film dissolves and opens when exposed to excessive amounts of water.

In another embodiment of the present invention, a method of manufacturing a hydro-degradable fluid absorbent is disclosed wherein the method comprising: assembling a top sheet formed by a hydro-degradable layer, an absorbent core with or without super absorbent polymer, a bottom sheet comprising of a tie layer which is coated on water-soluble film in entirety or in alternate pattern of strips of coat followed by moisture barrier coat in entirety or in alternate pattern of strips of coat respectively wherein the water-soluble film is towards the core and the coated moisture barrier coat towards the garment side or vice-versa with the tie layer sandwiched in between and bonding the top sheet and the bottom sheet with the absorbent core sandwiched in between by suitable industrial bonding techniques. FIG. 8(A-D) illustrates a sectional view of different types of pattern coatings on bottom sheet of the hydro-degradable fluid absorbent article.

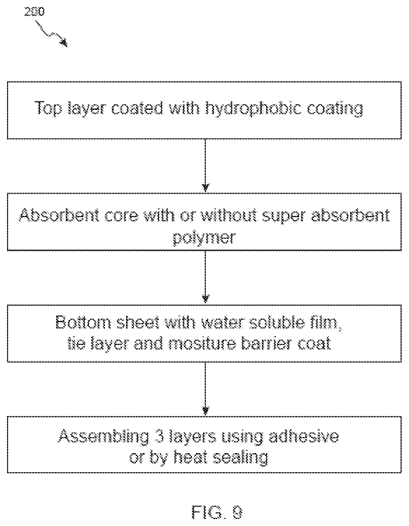

FIG. 9 represents a flowchart (200) illustrating method of manufacturing of the hydro-degradable fluid absorbent.

The method of manufacturing a hydro-degradable fluid absorbent further comprises of adding positioning glue on the back of the absorbent to which a release liner (108) is attached. FIGS. 2 and 3 depict the bottom sheet (106) with two solid strips of glue (112) to which release liner (108) is attached.

### EXAMPLES

### Example 1

A hydro-degradable fluid absorbent product's bottom sheet comprising at least one water soluble film characterized by a tie layer which is coated on water-soluble film followed by moisture barrier coat. The water soluble film is for example partially hydrolysed polyvinyl alcohol (PVOH, 70-85%). The moisture barrier layer is made up of polyvinylidene chloride (PVDC) to make it impervious to water. The tie layer is made of nitrocellulose based chemistry to improve the adhesion between PVOH and PVDC. Further, this tie layer acts as an anchoring layer for PVDC to PVOH, as PVDC possesses elastic and tie layer exhibits brittle characteristics, combination of both the materials gives synergistic effect and optimizes the elasticity of the coating material, assisting in faster disintegration post disposal. The tie layer is first coated onto the PVOH layer and then, PVDC layer is coated on top of the tie layer. To test the versatility of the tie layer, peel off test (in machine direction (MD) and cross direction (CD)) and moisture permeation test is conducted with and without tie layer present in the bottom sheet as described in paragraphs to and mentioned in Table 1.

The bottom sheet consisting of water soluble film of PVOH coated with moisture barrier layer of PVDC and with tie layer sandwiched in between has been used further to construct the hydro-degradable fluid absorbent in Ex-2 to Ex-8 with tie layer the coating is resulting in better adhesion to water soluble film reducing possibility of failure during use. As demonstrated by moisture permeation data below, it provides better barrier to moisture as compared to without tie layer coating.

TABLE 1

| Properties | With Tie layer | Without Tie layer |
|---|---|---|
| Peel off (MD and CD) | Passed | Failed |
| Moisture permeation (%) | 1% (in 6 hrs) | 25% (in 6 hrs) |

### Example 2

A hydro-degradable fluid absorbent comprising: a) a top sheet formed of hydro-degradable layer, wherein the top sheet along with the edges is coated with hydrophobic coating that covers 0-80% of the said sheet and 100% of its edges; b) an absorbent core, optionally with super absorbent polymer; c) a bottom sheet comprising at least one water soluble film characterized by a tie layer which is coated on water-soluble film followed by moisture barrier coat.

The bottom sheet is constructed in a way such that the water-soluble film is towards the adsorbent core and moisture barrier layer is towards the garment side with the tie layer sandwiched in between. The hydro-degradable fluid absorbent is constructed by bonding the top sheet and the bottom sheet with the absorbent core sandwiched in between using thermal bonding and/or hot melt adhesives and/or water soluble adhesives. The durability and flushability of constructed hydro-degradable fluid absorbent is conducted as described in paragraphs [072], [073] and [074] and mentioned in Table 2.

TABLE 2

| Properties | Ex-2 |
|---|---|
| Durability of Sanitary Pad (Pass or Fail) | Passed (upto 3 hrs) |
| Flushability of Sanitary Pad (Pass or Fail) | Passed |

### Example 3

A hydro-degradable fluid absorbent comprising: a) a top sheet formed of hydro-degradable layer, wherein the top sheet along with the edges is coated with hydrophobic coating that covers 0-80% of the said sheet and 100% of its edges; b) an absorbent core, optionally with super absorbent polymer; c) a bottom sheet comprising at least one water soluble film characterized by a tie layer which is coated on water-soluble film followed by moisture barrier coat.

The bottom sheet is constructed in a way such that the water-soluble film is towards the garment side and moisture barrier layer is towards the adsorbent core with the tie layer sandwiched in between. The hydro-degradable fluid absorbent is constructed by bonding the top sheet and the bottom sheet with the absorbent core sandwiched in between using thermal bonding and/or hot melt adhesives and/or water soluble adhesives. The durability and flushability of constructed hydro-degradable fluid absorbent is conducted as

described in paragraphs [072], [073] and [074] and mentioned in Table 3.

TABLE 3

| Properties | Ex-3 |
|---|---|
| Durability of Sanitary Pad (Pass or Fail) | Passed (upto 4 hrs) |
| Flushability of Sanitary Pad (Pass or Fail) | Passed |

### Example 4

A hydro-degradable fluid absorbent comprising: a) a top sheet formed of hydro-degradable layer, wherein the top sheet along with the edges is coated with hydrophobic coating that covers 0-80% of the said sheet and 100% of its edges; b) an absorbent core, optionally with super absorbent polymer; c) a bottom sheet comprising at least one water soluble film characterized by a tie layer which is coated on water-soluble film followed by moisture barrier coat.

The bottom sheet comprising of two sheets of water-soluble film characterized in that each film further comprises of a tie layer which is coated on water-soluble film followed by moisture barrier coat, such that the two sheets of film have water soluble layers facing towards each other, resulting in moisture barrier coat of one layer is towards the adsorbent core and of other layer is towards the garment side. Both inner and outer sheet are of same width, such that both sheets of the bottom sheet are sealed or bonded with top sheet, for construction of hydro-degradable fluid adsorbent. These two sheets of bottom sheet can either be boned together only at the edges or throughout the surface by applying thermal bonding and/or hot melt adhesives and/or water soluble adhesives. The hydro-degradable fluid absorbent is constructed by bonding the top sheet and the bottom sheet with the absorbent core sandwiched in between by using the techniques discussed above. The durability and flushability of constructed hydro-degradable fluid absorbent is conducted as described in paragraphs [072], [073] and [074] and mentioned in Table 4.

TABLE 4

| Properties | Ex-4 |
|---|---|
| Durability of Sanitary Pad (Pass or Fail) | Passed (up to 12 hrs) |
| Flushability of Sanitary Pad (Pass or Fail) | Failed |

Note:
The durability testing is stopped at 12 hrs

### Example 5

A hydro-degradable fluid absorbent comprising: a) a top sheet formed of hydro-degradable layer, wherein the top sheet along with the edges is coated with hydrophobic coating that covers 0-80% of the said sheet and 100% of its edges; b) an absorbent core, optionally with super absorbent polymer; c) a bottom sheet comprising at least one water soluble film characterized by a tie layer which is coated on water-soluble film followed by moisture barrier coat.

The bottom sheet comprising of two sheets of water-soluble film characterized in that each film further comprises of a tie layer which is coated on water-soluble film followed by moisture barrier coat, such that the two sheets of film have water soluble layers facing towards each other, resulting in moisture barrier coat of one layer is towards the adsorbent core and of other layer is towards the garment side. The width of the inner sheet is less than the width of the outer sheet, such that only one sheet (outer sheet) of the bottom sheet are sealed or bonded with top sheet, for construction of hydro-degradable fluid adsorbent. These two sheets of bottom sheet can either be boned together only at the edges or throughout the surface by applying thermal bonding and/or hot melt adhesives and/or water soluble adhesives. The hydro-degradable fluid absorbent is constructed by bonding the top sheet and the bottom sheet with the absorbent core sandwiched in between by using the techniques discussed above. The durability and flushability of constructed hydro-degradable fluid absorbent is conducted as described in paragraphs [072], [073] and [074] paragraph and mentioned in Table 5.

TABLE 5

| Properties | Ex-5 |
|---|---|
| Durability of Sanitary Pad (Pass or Fail) | Passed (up to 12 hrs) |
| Flushability of Sanitary Pad (Pass or Fail) | Passed |

Note:
The durability testing is stopped at12 hrs

Comparison of Ex-2, 3, 4 and 5: The durability of hydro-degradable fluid adsorbent with bottom sheet comprising of one or two sheets is compared in Table 6. It can be observed that hydro-degradable fluid adsorbent constructed with one bottom sheet resulted in maximum durability of 4 hrs, and while hydro-degradable fluid adsorbent constructed with two bottom sheets resulted in durability of 12 hrs.

TABLE 6

| Properties | Ex-2 | Ex-3 | Ex-4 | Ex-5 |
|---|---|---|---|---|
| Durability of Sanitary | Passed (up | Passed (up | Passed (up | Passed (up |
| Pad (Pass or Fail) | to 3 hrs) | to 4 hrs) | to 12 hrs) | to 12 hrs) |

The flushability of hydro-degradable fluid adsorbent with bottom sheet comprising of one or two sheets is compared in Table 7. It can be observed that hydro-degradable fluid adsorbent constructed with one bottom sheet passed the flushability criteria as mentioned in paragraph [073] and [074], and hydro-degradable fluid adsorbent constructed with two bottom sheets (width of the inner sheet is less than the width of the outer sheet) passed the flushability criteria, however hydro-degradable fluid adsorbent constructed with two bottom sheets (both inner and outer sheet are of same width) didn't passed the flushability criteria. This results from the fact that two bottom sheets are heat sealed with the top sheet which didn't allow the failure during flushing, while in case of Ex-5 only one bottom sheet is heat sealed and when flushed in the toilet bowl, the water soluble film dissolves and failure at the edge of interface of top and bottom sheet assist in flushability. This issue of non-flushable design can be overcome by using water-soluble adhesive during construction of hydro-degradable fluid adsorbent.

TABLE 7

| Properties | Ex-2 | Ex-3 | Ex-4 | Ex-5 |
|---|---|---|---|---|
| Flushability of Sanitary | Passed | Passed | Failed | Passed |
| Pad (Pass or Fail) | | | | |

### Example 6

A hydro-degradable fluid absorbent comprising: a) a top sheet formed of hydro-degradable layer, wherein the top sheet along with the edges is coated with hydrophobic coating that covers 0-80% of the said sheet and 100% of its edges; b) an absorbent core, optionally with super absorbent polymer; c) a bottom sheet comprising at least one water soluble film characterized by a tie layer which is coated on water-soluble film followed by moisture barrier coat.

The bottom sheet comprising at least one water soluble film is characterized by a tie layer which is coated in patterns of parallel strips with coated and uncoated zones on water-soluble film followed by moisture barrier coat on top of the tie layer such that the said coating is carried out on both sides of the film. The hydro-degradable fluid absorbent is constructed by bonding the top sheet and the bottom sheet with the absorbent core sandwiched in between using thermal bonding and/or hot melt adhesives and/or water soluble adhesives.

### Example 7

A hydro-degradable fluid absorbent comprising: a) a top sheet formed of hydro-degradable layer, wherein the top sheet along with the edges is coated with hydrophobic coating that covers 0-80% of the said sheet and 100% of its edges; b) an absorbent core, optionally with super absorbent polymer; c) a bottom sheet comprising at least one water soluble film characterized by a tie layer which is coated on water-soluble film followed by moisture barrier coat.

The bottom sheet comprising at least one water soluble film characterized by a tie layer which is coated as alternate pattern of strips of coat on water-soluble film followed by moisture barrier coat on top of coated alternate strips of tie layer and the other side of the film is coated with a tie layer in entirety, wherein, the tie-layer coated in entirety side of water-soluble film is towards the core and the coated moisture barrier layer towards the garment side and vice-versa. The hydro-degradable fluid absorbent is constructed by bonding the top sheet and the bottom sheet with the absorbent core sandwiched in between using thermal bonding and/or hot melt adhesives and/or water soluble adhesives.

### Example 8

A hydro-degradable fluid absorbent comprising: a) a top sheet formed of hydro-degradable layer, wherein the top sheet along with the edges is coated with hydrophobic coating that covers 0-80% of the said sheet and 100% of its edges; b) an absorbent core, optionally with super absorbent polymer; c) a bottom sheet comprising at least one water soluble film characterized by a tie layer which is coated on water-soluble film followed by moisture barrier coat.

The bottom sheet comprising two water soluble film is characterized by a tie layer which is coated in patterns of parallel strips with coated and uncoated zones on water-soluble film followed by moisture barrier coat on top of the tie layer such that the said coating is carried out on both sides of the film. The hydro-degradable fluid absorbent is constructed by bonding the top sheet and the bottom sheet with the absorbent core sandwiched in between using thermal bonding and/or hot melt adhesives and/or water soluble adhesives. All aforesaid embodiments can also be constructed using ultrasonic bonding.

To test the performance of different embodiments of absorbent product as aforesaid the following tests were carried out and the results recorded therein;

Peel off or Tape test: To test the versatility of tie layer, peel off test or tape test is conducted using a modified method of ASTM D3359. This test method is used to evaluate whether the adhesion i.e. molecular attraction and mechanical bonds between a coating and its substrate is adequate for the required application. Two test methods are available to evaluate the adhesion such as Method A and Method B.

Test Method A: An X-cut is made through the film to the substrate, pressure-sensitive tape is applied over the cut and then removed, and adhesion is assessed qualitatively on a 0 to 5 scale. Test Method B: A lattice pattern with either six or eleven cuts in each direction is made through the film to the substrate, pressure-sensitive tape is applied over the lattice pattern and then removed, and adhesion is assessed qualitatively on a 0 to 5 scale. Test Method A is primarily intended for use in the field while Test Method B is more suitable for use in laboratory or shop environments. As the bottom sheet consist of water soluble film in the range of 10-40 GSM with barrier coating of 7-12 GSM and the film is very soft and flexible, and sensitive to moisture along with low coat weight, the cuts with blades was not done and the standard method is modified as per requirement.

Procedure:
After complete curing of the coating on the films, this test method is conducted.
An area free of blemishes and minor surface imperfections is selected. The area chosen for testing shall be clean and dry.
Before initiation of testing, two complete laps of tape from the roll are removed and discarded. An additional length at a steady (that is, not jerked) rate is removed and a piece about 20 mm width×50 mm long is cut.
The center of the tape is placed at selected area with the tape running in the same direction (Machine direction) as the smaller angles. The tape is smoothened into place by finger in the area of the incisions taking care not to entrap air under the tape. The tape is rubbed firmly over the surface with the pressure application device until the color is uniform in appearance. This indicates good, uniform contact between the tape's adhesive and the coating surface.
Within 90±30 s of application, the tape is removed by seizing the free end and pulling it off rapidly (not jerked) back upon itself at as close to an angle of 180° as possible.
The area after peel off is inspected for removal of coating from the substrate.
Same procedure is also done on cross direction of coating to check the adhesion on the substrate.

Moisture Permeation test: The moisture permeation test is conducted on the sample (bottom sheet of the hydro-degradable fluid absorbent) to evaluate its barrier capacity. This test helps in understanding the moisture permeation rate over time. This test is conducted by pouring the synthetic blood and observing the moisture permeation through the film periodically. The synthetic blood is prepared as per ISO 16603. The defects in the sample usually form in the sequence of initially forming micro-droplets followed by the formation of wrinkles and finally, moisture permeates. The sample size taken for the experiment is 150 mm wide×300 mm long. First, the sample is plated on the glass plate without any wrinkles and taped at the edges. Then the synthetic blood is applied in a droplet form throughout the sample evenly with the help of a syringe. The glass plate is covered with a transparent acrylic tray to avoid evaporation of the blood. Then the sample is observed closely every hour of the test. The moisture permeation values are reported in the percentage. The test is concluded at the 12-hour mark or when the moisture permeation is more than 90% of the total area of the sample whichever is earlier. The test method is used to understand which design has a better moisture barrier (i.e., low moisture permeation).

Durability test: The durability test is performed to understand the duration of time the sample (hydro-degradable fluid absorbent) can meet its performance requirements. The sample should have enough absorbency capacity and the bottom film should have enough strength to hold the liquids during use. The test is carried out with the synthetic blood prepared as per ISO 16603 by dropping it on the hydro-degradable fluid absorbent material and observing the leaks at the bottom side periodically. The volume of blood taken and added to the sample is 15 mL. Initially, 5 mL is added to the absorbent to start the experiment then subsequent 5 mL is added every hour till 15 mL. The bottom side is observed every hour to check the condition and whether any damages on the bottom sheet or any leaks through it. The experiment is conducted for 12 hours or till any leaks or damages are observed on the bottom sheet, whichever is earlier.

Flushability: A. Toilet bowl clearance

The purpose of the test is to evaluate the sample which can dispose of through the toilet bowl into the drains or sewer, and not cause any clogs in the toilet pipeline. It is required to have the toilet setup with the 4.5-litre capacity flush tank (widely used volumes) for the experiment. The sample is simulated as being used before being disposed of in the toilet bowl, to represent typical product use. The 15 mL of synthetic blood is poured onto the absorbent and kept for 6 hours before it was disposed of into the toilet bowl. First, ensured that the flush volume is 4.5 litres (+/−0.2 litres). Then the toilet bowl is flushed to clear the debris that settled in it. The sample is placed in the bowl and flushed then observed whether the whole of the samples cleared the bowl and water trap pipe and entered into the drainline and also observed whether any surcharges happened in the bowl. In case the sample does not clear the bowl and the water trap pipe, i.e., the product is not visible in the drainline, wait for the flush tank to fill and repeat the flushing. When the whole sample entered the drain line, the total number of flushes required is recorded. The pass criteria are that greater than 95% of the products tested out of 40 samples clear the bowl in no more than 2 flushes, and any surcharging of the bowl is below the rim on 100% of the tests. (As per Fine to Flush, UK Water Industry Specifications)

B. Drainline clearance test: The purpose of the test is to ensure the product passes through the household drainline system without any problem associated with settling out or snagging. The household pipeline is the fowl pipe connected to the public sewer line. It is usually 100 mm in diameter and the slope of the pipeline would be 1:80. The length of the pipeline connected from the toilet system to the sewer line would be 20 m. It is required to have the toilet setup with the 4.5-litre capacity flush tank (widely used volumes) for the experiment. The sample is simulated as being used before being deposited in the WC bowl, to represent typical product use. The 15 mL of synthetic blood is poured onto the pad and kept for 6 hours before it is disposed of into the toilet bowl. First, ensured that the flush volume is 4.5 litres (+/−0.2 litres). Then the toilet bowl is flushed to clear the debris that settled in it. Initially, the sample is placed in the bowl and performed the flush. Once the sample entered the drainline, started to observe the travel distance of the sample along the drainline. If the sample does not clear the bowl and the water trap pipe, wait for the flush tank to fill and repeat the flushing till it enters the drainline. When the whole of the sample entered the drain, the distance travelled by the sample along the drainline was measured. Waited for the flush tank to fill and continued the consecutive flushes till the samples passed the 20 m drainline or the samples stayed stationary for consecutive 3 flushes. If, after five flushes, the sample didn't travel a distance of 20 m from the toilet connection into the drainline, conclude the test. The pass criteria are when tested by the method specified above, in at least 95% of the 40 tests, the product shall travel a distance of at least 20 m along the drainline, within 5 consecutive flushes; and not remain stationary for more than 3 consecutive flushes. (As per Fine to Flush, UK Water Industry Specifications)

What is claimed is:

1. A hydro-degradable fluid absorbent comprising:
a) a top sheet formed of hydro-degradable layer, wherein the top sheet along with the edges is coated with hydrophobic coating that covers 0-80% of the said sheet and 100% of its edges;
b) an absorbent core, optionally with super absorbent polymer;
c) a bottom sheet comprising at least one water soluble film characterized by a tie layer which is coated on water-soluble film followed by moisture barrier coat.

2. The hydro-degradable fluid absorbent as claimed in claim 1, wherein the bottom sheet is constructed in a way such that the water-soluble film is towards the adsorbent core and moisture barrier layer is towards the garment side and vice-versa with the tie layer sandwiched in between.

3. The hydro-degradable fluid absorbent as claimed in claim 1, wherein the bottom sheet comprising at least one water soluble film characterized by a tie layer coated on both sides of water-soluble film followed by moisture barrier coat on both sides with variation of thickness on each side.

4. The hydro-degradable fluid absorbent as claimed in claim 1, wherein the bottom sheet comprising at least one water soluble film is characterized by a tie layer which is coated as alternate pattern of strips with coated and uncoated zones on water-soluble film followed by moisture barrier coat on top of coated alternate strips the tie layer such that the said coating is carried out on both sides of the film characterized in that the coating on other side of the film is done on the area which is uncoated on the opposite side.

5. The hydro-degradable fluid absorbent as claimed in claim 1, wherein the bottom sheet comprising at least one water soluble film characterized by a tie layer which is coated as alternate pattern of strips of coat on water-soluble film followed by moisture barrier coat on top of coated alternate strips of tie layer and the other side of the film is coated with a tie layer in entirety, wherein, the tie-layer coated in entirety side of water-soluble film is towards the core and the coated moisture barrier layer towards the garment side and vice-versa.

6. The hydro-degradable fluid absorbent as claimed in claim 1, wherein the bottom sheet comprising at least one water soluble film characterized by a tie layer which is coated as alternate pattern of strips of coat on water-soluble film followed by moisture barrier coat on top of coated alternate strips of tie layer; the other side of the water soluble film is coated with a tie layer in entirety, followed by moisture barrier coat, wherein the tie layer coated alternate pattern of strips of coat side of the water-soluble film is towards the core and the coated moisture barrier layer towards the garment side and vice-versa.

7. The hydro-degradable fluid absorbent as claimed in claim 1, wherein said at least one water soluble film of the bottom sheet comprises two sheets of water soluble film.

8. The hydro-degradable fluid absorbent as claimed in claim 1, wherein the bottom sheet comprising of two sheets of water-soluble film characterized in that each film further comprises of a tie layer which is coated on water-soluble film followed by moisture barrier coat, such that the two sheets of film have water soluble layers facing towards each other, resulting in moisture barrier coat of one layer is towards the adsorbent core and of other layer is towards the garment side.

9. The hydro-degradable fluid absorbent as claimed in claim 1, the bottom sheet comprising of two sheets of water-soluble film characterized in that either both inner and outer sheet are of same width or the width of the inner sheet is less than the width of the outer sheet, such that both sheets or only one sheet (outer sheet) of film of the bottom sheet are sealed or bonded with top sheet respectively by using any of the industrial bonding techniques such as thermal bonding, hot melt adhesives, water soluble adhesives or ultrasonic bonding for construction of hydro-degradable fluid adsorbent.

10. A method of manufacturing a hydro-degradable fluid absorbent comprising:
assembling, a) a top sheet formed by a hydro-degradable layer, b) an absorbent core optionally with super absorbent polymer, c) a bottom sheet comprising at least one water soluble film characterized by a tie layer which is coated on water-soluble film followed by moisture barrier coat and bonding the top sheet and the bottom sheet with the absorbent core sandwiched in between by using any of the industrial bonding techniques such as thermal bonding, hot melt adhesives, water soluble adhesives and ultrasonic bonding.
